(19) United States
(12) Patent Application Publication
(10) Pub. No.: US 2012/0109290 A1
(43) Pub. Date: May 3, 2012

(54) PROSTHESIS FOR REPAIRING HEART VALVES

(76) Inventor: Carlo Antona, Assago MI (IT)

(21) Appl. No.: 13/266,890

(22) PCT Filed: Apr. 29, 2009
(86) PCT No.: PCT/IT2009/000189

§ 371 Date: Jan. 17, 2012

(51) Int. Cl. A61F 2/24

(52) U.S. Cl. 623/2.12

(57) ABSTRACT

The present invention relates to a prosthesis and a method for extending the valve leaflets (2a, 2b, 2c) of a heart valve (2) using the prosthesis (1). The prosthesis (1) comprises at least two strip elements (3-5), each of which has two opposed ends (3a, 3b, 4a, 4b, 5a, 5b) and two opposed edges (3c, 3d, 4c, 4d, 5c, 5d). The prosthesis is characterized in that the at least two strip elements (3-5) are connected at their ends (3a, 3b, 4a, 4b, 5a, 5b) to form cusps and in that it comprises additional plate elements (9-11) connected to said strip elements (3-5) at said cusp points.

The present invention relates to a prosthesis for repairing heart valves, particularly the aortic valve.

Semilunar aortic and pulmonary valves have the function of preventing the blood from flowing back into the heart after being introduced into arterial circulation. Impaired function of these valves due to congenital defects or degeneration, in the form of stenosis and/or regurgitation, may have serious consequences for the function of the heart muscle, and therefore it may sometimes be necessary to replace the valve or to repair it.

Biological or mechanical prostheses are used for the replacement of the valve, whereas in repairing the valve the geometry and integrity of the leaflets are restored in order to achieve normal valve function.

In particular, in the case of aortic valve regurgitation, the operation aims to restore the correct coaptation of the valve leaflets, thus providing complete continence of the valve.

Different methods may be used for this purpose, as follows:

Subcommissural plasty, in which the subcommissural triangle is narrowed

Narrowing of the valve annulus using prostheses or rings

Adjusting the size of the sinotubular junction, using prostheses or by plication of the junction

Correction of any prolapse of one or more valve leaflets, using plication or resection methods

Repair of any damage to the leaflets due to degeneration or infection, by direct repair or by application of patches of homologous or heterologous material

Extension of the valve leaflets by application of pericardial tissue or prosthetic material.

In some cases it is necessary or preferable to avoid the complete replacement of the valve, for example if the patient is very elderly or very young, or when there is a risk of infection, or when the poor coaptation is due to a partial loss of tissue while the rest of the valve apparatus has normal morphology.

In all this situations, it is preferable to use conservative methods to avoid prosthetic replacement.

Each of the aforementioned methods is used according to the type of impairment present in the valve.

In particular, in the case of extension of the valve leaflets, this extension method is preferred when the reflux is due to the fact that only one or more of the valve leaflets has an insufficient surface area to ensure correct coaptation. The method consists in suturing a strip of tissue, usually pericardial tissue, to the free margin of the valve leaflet, in order to increase the contact area between the leaflets, thus increasing their coaptation surface area.

One of the limitations of this method is the fact that the surgeon has to cut off a “strip” (or “leaf”) of adequate size during the operation in order to suture the strip to the valve leaflet to be extended; in practice, the surgeon has to be skilful enough to provide an extension of the valve leaflet whose length is equal to that of the leaflet concerned and whose depth is sufficient to ensure coaptation with the other leaflets.

The object of the present invention is therefore to provide a heart valve repair prosthesis, and particularly a prosthesis for extending the free leaflets of the aortic valve.

The present invention makes it possible to provide a heart valve repair prosthesis which is ready for use without the need for the surgeon to act on it to make it compatible with the aortic valve to be repaired. This leads to a higher success rate of the surgical operation, since the prosthesis made according to the invention is less affected by the degree of skill of the surgeon.

The characteristics and advantages of the present invention will be made clear by the following detailed description of a practical embodiment illustrated by way of non-limiting example in the appended drawings, in which:

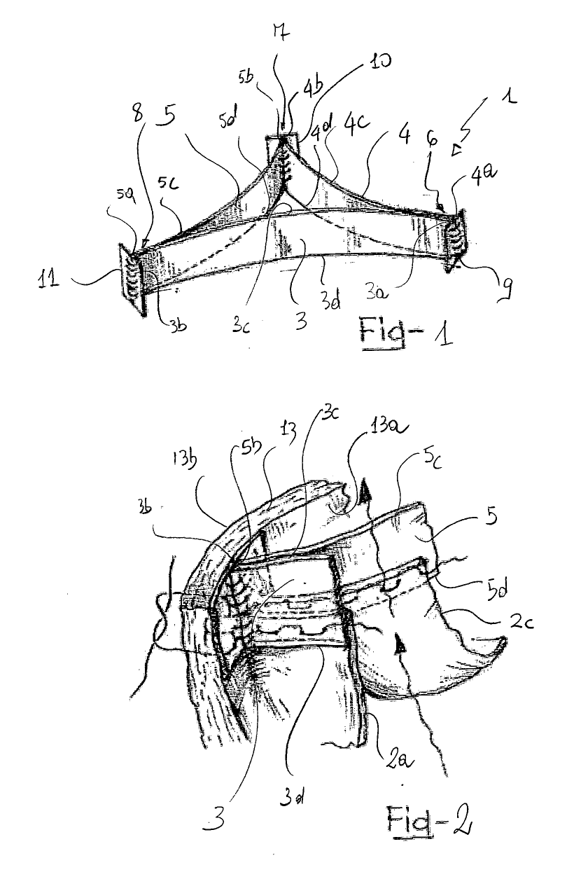

FIG. 1 is a perspective view of one embodiment of the prosthesis according to the present invention;

FIG. 2 is a perspective view in partial cross section of an artery and of a heart valve after the insertion of the prosthesis of the type shown in FIG. 1;

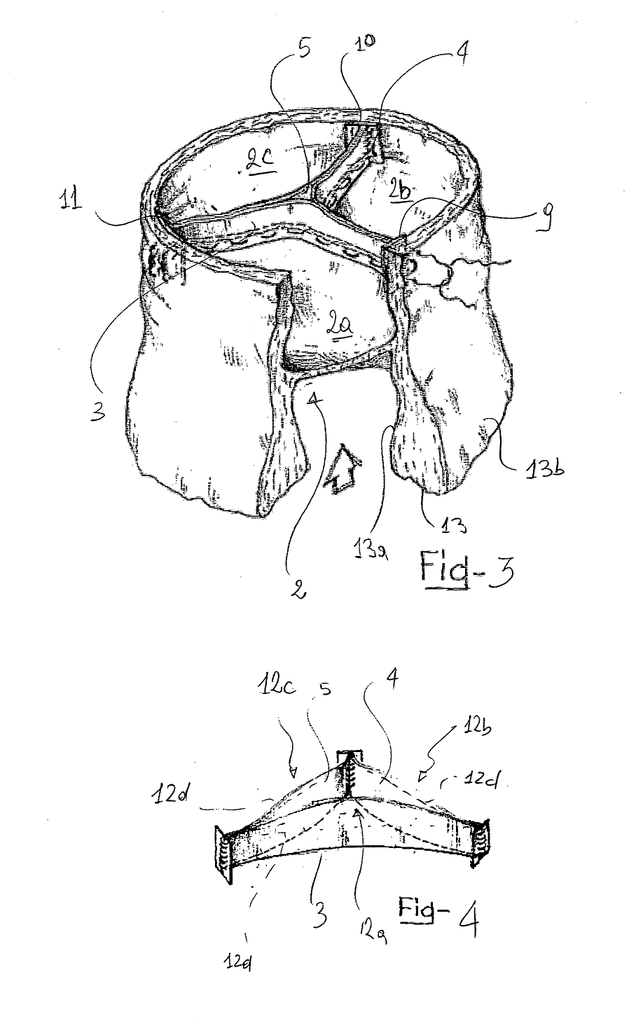

FIG. 3 is a perspective view in partial cross section in a closed configuration of the artery and of the heart valve after the insertion of the prosthesis of the type shown in FIG. 1;

FIG. 4 shows another embodiment of the prosthesis according to the present invention.

It should be noted that, although a prosthesis for a tricuspid heart valve is shown in the appended drawings, this prosthesis can also be applied in the case of a bicuspid heart valve, as will be clear from the remainder of the present description.

With reference to the appended drawings, the number 1 indicates the whole of a prosthesis for a heart valve 2, preferably an aortic valve.

The prosthesis 1 comprises three strip elements 3, 4 and 5, each of which has two opposed ends and two opposed edges.

For example, the strip element 3 has ends 3a and 3b and edges 3c and 3d. The same applies to the other strip elements 4 (with ends 4a and 4b and edges 4c and 4d) and 5 (with ends 5a and 5b and edges 5c and 5d).

In particular, in the case of a tricuspid heart valve, the prosthesis 1 comprises three strip elements 3-5, interconnected in such a way that:
the first end 3a of the first strip element 3 is connected to the first end 4a of a second strip element 4, and that
the second end 3b of the first strip element 3 is connected to a first end 5a of a third strip element 5, and that
the second end 4b of the second strip element 4 is connected to the second end 5b of the third strip element 5.

In particular, the strip elements 3-5 are connected at their ends to form the cusps 6-8 respectively.

It should be noted that the term “cusp” is used in the following part of the description to denote the vertex or point which is formed when the ends of the strip elements 3-5 of the prosthesis 1 are joined.

The prosthesis 1 comprises additional plate elements 9-11 connected to the strip elements 3-5 at the aforesaid cusp points.

That is to say, the ends 3a, 3b, 4a, 4b, 5a and 5b of the strip elements are connected not only to each other but also to corresponding plate elements.

In particular, each plate element 9-11 is positioned at each cusp 6-8 in such a way that it is connected to the strip elements.

For example, the ends 3b-5b of the corresponding strip elements 3 and 5 are interconnected and are also connected to the plate element 11. Similarly, the ends 2a-4a are connected to the plate element 9 and the ends 4b-5b are connected to the plate element 10.

Each plate element 9-11 has two pairs of opposed sides and has a cross section in plan view which is quadrangular, and preferably rectangular.

It should be noted that each plate element 9-11 is connected to each cusp (formed by the connection of the ends of the strip elements 3-5) along a connection region which extends between two opposed sides of said pair.

Preferably, this connection region extends between two opposed sides in a direction of extension substantially parallel to the direction of extension of the sides of the other pair.

Additionally, the connection region lies on a median line of the plate element.

For example, the ends 3b-5b of the corresponding strip elements 3 and 5 are interconnected and are also connected to the plate element 11 along a connection region which extends between the two opposed sides of the plate element 11 along a median line.

In a preferred embodiment, the extension of the ends 3a-3b, 4a-4b and 5a-5b of the strip elements 3-5 ranges from about 0.8 cm to about 1.4 cm, and is preferably equal to about 1 cm.

As regards the extension of the edges 3c-3d, 4c-4d and 5c-5d of the strip elements 3-5, this must be such that the prosthesis 1 can be inserted into an artery 13 having a specific diameter, where the heart valve 2 to be repaired is located. In particular, when the possible values of the diameter of the artery 13 are known, the prosthesis 1 must be made with extensions of the edges 3c-3d, 4c-4d and 5c-5d such that they are compatible with the aforesaid values.

Consequently, the extensions of the edges 3c-3d, 4c-4d and 5c-5d are such that the prosthesis 1, in an inoperative configuration, in other words before placing in the proximity of the valve 2 to be repaired, can be inscribed in a circle having a diameter ranging from about 1.5 cm to about 3.3 cm.

In particular, it is useful to note that the imaginary circle in which the prosthesis 1 can be inscribed passes through the cusp points, in other words through the corresponding ends of the strip elements which are interconnected to form the cusps of the prosthesis.

According to a preferred aspect of the present embodiment, the connection of the strip elements 3-5 at the corresponding ends 3a-3b, 4a-4b and 5a-5b to form the cusps 6-8 and the connection to each plate element 9-11 are made by one or more stitches formed, for example, with suture wire.

It should also be noted that both the strip elements 3-5 and the plate elements 9-11 are composed of biocompatible material such as pericardium or synthetic material.

With particular reference to FIG. 1, it should be noted that the directions of extension of all the pairs of edges 3c-3d, 4c-4d and 5c-5d of the strip elements 3-5 are substantially parallel to each other. In this specific illustration, therefore, the strip elements 3-5 take the form of rectangular bands.

Alternatively, and with reference to FIG. 4 which shows another embodiment of the prosthesis 1, it may be noted that at least one edge of each pair of edges 3c-3d, 4c-4d and 5c-5d of the strip elements 3-5 extends along at least one portion 12a, 12b, 12c which lies along the imaginary straight line 12d joining the vertices of the two ends.

In particular, the portion 12a, 12b, 12c is such that it lies on the imaginary straight line 12d remotely from the other edge.

In a preferred embodiment, the portion 12a, 12b, 12c is convex.

For example, the strip element 3 has an edge 3c comprising the portion 12a which lies on the imaginary straight line joining the ends 3a and 3b, this portion 12a being remote from the other edge 3d.

Additionally, there may be combinations of the convex and parallel configurations. In particular, a portion of the lower edge may have a convex shape.

With reference to FIGS. 2 and 3, which show in partial section a tricuspid heart valve to be repaired, a method will now be described for implanting the prosthesis 1 in order to increase the coaptation of the valve leaflets 2a, 2b, 2c of the valve 2 located inside the artery 13.

It should be noted that the arrow shown in the aforesaid FIGS. 2 and 3 indicates the direction of the blood flow, while the artery 13 has an inner wall 13a and an outer part 13b, the blood flowing over the inner wall 13a.

Consequently, the present invention also relates to a method of extending the valve leaflets 2a, 2b, 2c of a heart valve 2, particularly those of the aortic valve to be repaired, this method comprising:
providing a prosthesis 1 of the type described above;
opening an artery 13 transversely with respect to its longitudinal direction of extension, the opening preferably being made along at least three quarters of the circumference, the valve 2 to be repaired being located in the artery 13;
measuring the diameter of the artery 13 at the opening point;
selecting the prosthesis 1 according to the diameter of the artery 13;
inserting the selected prosthesis 1 into the open artery;
suturing each plate element 9-11 to the upper point of each of the aortic commissures;
superimposing the edge, for example the edge 3c, 4c and 5c of each of the strip elements 3-5, on a corresponding free end of the leaflet 2a, 2b, 2c of the valve 2 to be repaired;
suturing each strip element 3-5 to the corresponding free end of the leaflet 2a, 2b, 2c of the valve 2 to be repaired in such a way that the edges 3d, 4d, 5d of each strip element 3-5 lie on top of the valve leaflets 2a, 2b, 2c, and, in a closed configuration of the valve to be repaired (as shown in FIG. 3 for example), the other edges 3c, 4c, 5c meet to close the valve 2 by coaptation, this coaptation of the other edges 3c, 4c, 5c taking place over at least 60% of the extension of the end 3a-3b, 4a-4b, 5a-5b of each strip element 3-5.

In other words, the edges 3c, 4c, 5c of each strip element 3-5 are in surface contact with each other over at least 60% of the extension of the ends 3a-3b, 4a-4b, 5a-5b of each strip element 3-5.

On the other hand, the edges 3d, 4d, 5d are superimposed on the valve leaflets 2a, 2b, 2c of the valve 2. The superimposition is such that the edges 3d, 4d, 5d are positioned on the outer surfaces of the leaflets 2a, 2b, 2c with reference to the direction of blood flow.

In particular, the prosthesis 1 is thus positioned in such a way that:
the edges 3c, 4c, 5c of the corresponding strip elements 3-5 are free and meet to close the valve 2 by coaptation when the valve is in the closed configuration, and that
the edges 3d, 4d, 5d of the corresponding strip elements 3-5 are placed in contact with the valve leaflets 2a, 2b, 2c, preferably being placed on top of the contacted leaflets with reference to the direction of the blood flow.

Assuming that the extension of the ends is 1 cm, the coaptation between the strip elements extends for about 0.6 cm.

Owing to the aforesaid characteristic, when the valve 2 is in the closed configuration the strip elements 3-5 of the prosthesis 1 interact to impede the flow of blood through the valve 2, thus preventing harmful regurgitation of blood.

As regards the plate elements 9-11, these are sutured to the upper points of the three commissures (in the case of a tricuspid aortic valve) on the inner surface 13a of the artery 13.

The upper points of the commissures of the three valve leaflets 2a, 2b, 2c can easily be identified by the surgeon.

It should be noted that the plate elements 9-11 are sutured with stitches formed on the outer wall 13b of the artery 13. These stitches are positioned outside the median region along which is located the region of connection of the ends of the two corresponding strip elements.

It should be noted that, before the strip elements 3-5 are sutured to the corresponding valve leaflets 2a, 2b, 2c, attention must be paid to the parallelism between the edges of the strip elements and the valve leaflets 2a, 2b, 2c. If this condition is met, the effectiveness of the coaptation can be guaranteed.

Alternatively, the method of extending the valve leaflets 2a, 2b, 2c of the heart valve 2 may include the superimposition of the edges 3d, 4d, 5d on the valve leaflets 2a, 2b, 2c of the valve 2, in such a way that these edges 3d, 4d, 5d are positioned on the inner surfaces of the leaflets 2a, 2b, 2c with reference to the direction of blood flow.

Additionally, according to what is shown specifically in FIGS. 2 and 3, it should be noted that the suturing of the strip elements 3-5 to the leaflets 2a, 2b, 2c is carried out using stitches positioned transversely to the direction of blood flow.

If the prosthesis 1 has to be associated with a bicuspid heart valve, the prosthesis (not shown in the drawings) is composed of only two strip elements (for example, strip elements 3 and 4) whose respective ends (the ends 3a-3b and 4a-4b) are interconnected (for example, the end 3a is connected to the end 4a and the end 3b is connected to the end 4b) to form cusps, and each cusp is in turn connected to a plate element (for example, plate elements 9 and 10).

The method for repairing a bicuspid valve is similar to that described above for a tricuspid valve.

Clearly, a person skilled in the art can make numerous modifications and variations to the embodiment described above, in order to meet contingent and specific requirements, such modifications and variations being contained within the scope of protection of the invention as defined in the following claims.

1.-17. (canceled)

18. A prosthesis (1) suitable for extending the valve leaflets (2a,2b,2c) of a heart valve (2) comprising at least two strip elements (3-5), each of which has two opposed ends (3a, 3b, 4a, 4b, 5a, 5b) and two opposed edges (3c, 3d, 4c, 4d, 5c, 5d), characterized in that said at least two strip elements (3-5) are connected at their respective ends (3a, 3b, 4a, 4b, 5a, 5b) to form cusp points, the extension of the end (3a, 3b, 4a, 4b, 5a, 5b) of each of said strip elements (3-5) being in the range from 0.8 cm to 1.4 cm in order to ensure coaptation with the valve leaflets (2a,2b,2c) and said plate elements (9-11) connected to said strip elements (3-5) at said cusp points.

19. A prosthesis for a heart valve according to claim 18, comprising three strip elements (3-5) in which the first end (3a) of the first strip element (3) is connected to the first end (4a) of a second strip element (4) and the second end (3b) of the first strip element (3) is connected to a first end (5a) of a third strip element (5) and the second end (4b) of the second strip element (4) is connected to the second end (5b) of the third strip element (5).

20. A prosthesis for a heart valve according to claim 18, in which a plate element (9-11) is positioned at each cusp formed by the connection of the corresponding ends (3a, 3b, 4a, 4b, 5a, 5b) of the strip elements (3-5).

21. A prosthesis for a heart valve according to claim 18, in which said cusp points formed at the corresponding ends (3a, 3b, 4a, 4b, 5a, 5b) of the strip elements (3-5) can be inscribed in a circle having a diameter in the range from 1.5 cm to 3.3 cm.

22. A prosthesis for a heart valve according to claim 18, in which said strip elements (3-5) are connected at their corresponding ends (3a, 3b, 4a, 4b, 5a, 5b) to form cusps by means of one or more stitches in each case.

23. A prosthesis for a heart valve according to claim 18, in which the direction of extension of both of the opposed edges (3c, 3d, 4c, 4d, 5c, 5d) of each of said strip elements (3-5) is substantially parallel.

24. A prosthesis for a heart valve according to claim 18, in that at least one portion (12a, 12b, 12c) of the length of at least one edge (3c, 3d, 4c, 4d, 5c, 5d) of each of said strip elements (3-5) lies on the imaginary straight line (12d) joining the vertices of the two ends (3a, 3b, 4a, 4b, 5a, 5b) in a position remote from the other, opposed edge.

25. A prosthesis for a heart valve according to claim 18, in which said plate elements (9-11) have a quadrangular section in plan view.

26. A prosthesis for a heart valve according to claim 18, in which said plate elements (9-11) have two pairs of opposed sides and each of said plate elements (9-11) is connected to a corresponding cusp formed by the connection of the ends of the strip elements (3-5) along a connection region extending between two opposed sides of said pair of sides.

27. A prosthesis for a heart valve according to claim 26, in which the connection region extends between two opposed sides along a direction of extension substantially parallel to the direction of extension of the sides of the other pair.

28. A prosthesis for a heart valve according to claim 27, in which the connection region is positioned on a median line of each plate element (9-11).

29. A prosthesis for a heart valve according to claim 18, in which said strip elements (3-5) and said plate elements (9-11) are made from biocompatible material.

30. A method for extending the valve leaflets (2a, 2b, 2c) of a heart valve (2), comprising the steps of:
providing a prosthesis (1) of the type defined in claim 18;
wherein it comprises the steps of:
opening an artery (13) transversely with respect to its longitudinal direction of extension, the valve (2) to be repaired being located in said artery (13), in such a way that said prosthesis (1) is inserted into the open artery;
superimposing an edge (3c, 3d, 4c, 4d, 5c, 5d) of each of the strip elements (3-5) on a corresponding free end of the leaflet (2a, 2b, 2c) of the valve (2) to be repaired;
suturing each plate element (9-11) to the upper point of each of the aortic commissures;
suturing each strip element (3-5) to the corresponding free end of the leaflet (2a, 2b, 2c) of the valve (2) to be repaired in such a way that an edge (3d, 4d, 5d) of the pair of edges (3c, 3d, 4c, 4d, 5c, 5d) of each strip element (3-5) lies on top of the valve leaflets (2a, 2b, 2c), and, in a closed configuration of the valve (2) to be repaired, the other edge (3c, 4c, 5c) of the pair of edges (3c, 3d, 4c, 4d, 5c, 5d) meets to close the valve (2) by coaptation, this coaptation taking place over at least 60% of the extension of the end (3a-3b, 4a-4b, 5a-5b) of each strip element (3-5).

31. A method according to claim 30, comprising the additional steps of:
measuring the diameter of the artery (13) at the opening point;
selecting said prosthesis (1) according to the diameter of the measured artery.

32. A method according to claim 30, in which the suturing of the edge (3d, 4d, 5d) of the pair of edges (3c, 3d, 4c, 4d, 5c, 5d) of each strip element (3-5) which lies on top of the valve leaflets (2a, 2b, 2c) is carried out by means of stitches positioned transversely with respect to the direction of blood flow.

33. A method according to claim 30, in which said step of opening the artery (13) transversely with respect to its longitudinal direction of extension is carried out along at least three quarters of the circumference of the artery.
